(19) United States
(12) Patent Application Publication
(10) Pub. No.: US 2005/0074510 A1
(43) Pub. Date: Apr. 7, 2005

(54) Topical preparations for use in treatment of anorectal disease

(76) Inventor: Paul J. Bobrowski, Scottsdale, AZ (US)

Correspondence Address: ELLIS & VENABLE, PC, 101 NORTH FIRST AVE., SUITE 1875, PHOENIX, AZ, 85003, US

(21) Appl. No.: 10/678,990

(22) Filed: Oct. 4, 2003

(51) Int. Cl. A61K 35/78

(52) U.S. Cl. 424/779

(57) ABSTRACT

The invention herein describes an improved approach to managing hemorrhoids by combining current approaches of topical anesthetics, vasoconstrictors, ant-inflammatories, protectants, anti-pruretics, astringents, keratolytics, anticholinergics, cicatrizants, and antimicrobials with Croton species material. The botanical extracts are concentrated biologically active materials and thus provide enhanced therapeutic benefit. The Croton species components add a breadth and immediacy to the termination of neurogenic responses that mediate, sustain, and prolong the symptoms and underlying pathology.

### BACKGROUND OF THE INVENTION

This application claims benefit of U.S. Provisional Application having the same title, serial number TBD filed on Oct. 5, 2002.

### FIELD OF THE INVENTION

Hemorrhoids are ubiquitous and specific to Homo sapiens, with the majority of people, both males and females, experiencing symptoms in their lifetimes. They are broadly categorized based upon their location relative to the dentate line of the anal canal: those which occur above the dentate are classified internal and those below as external. The dentate distinguishes two different skin types and whereas the skin above the dentate line is not sensitive to pain, the skin below is and thus external hemorrhoids are generally painful.

Thrombosed external hemorrhoids are those in which the blood has clotted resulting in pain and resulting inflammation in proximal pain sensitive skin. Inflammation of anorectal tissues is a complicating factor that enhances the severity of hemorrhoids. The patient often suffers from chronic pain and itching. The inflamed tissues become sensitized to the application of medicines intended to treat the problem and this sensitization can actually cause the medicines to worsen the problem that they are intended to treat.

Hemorrhoids are not limited to the modern era but common throughout history. In the 4^{th} century BC, in his writings, the Greek philosopher Hippocrates described appropriate treatments including cauterization, surgical removal, chemical treatment and ligation, which are much the same as, albeit refined, treatments employed today. Surgery sometimes does not eliminate the continuing tendency of these tissues to become inflamed and in most cases offers only a temporary solution to the problem. Thus, the cycle of inflammation, discomfort and sensitization repeats itself even after surgery.

However, most hemorrhoidal conditions are not so severe as to require surgery and are addressed through topical formulas. These preparations typically contain a vasoconstrictive agent(s) to shrink the venous dilations (the underlying cause of hemorrhoids), a protectant(s) to shield the sensitized tissues and in some cases, an anti-itch agent. Unfortunately, many patients have reported that these products do not provide complete relief from the anorectal inflammation accompanying hemorrhoids.

Substance P (SP) is a peptide that acts on specific post-synaptic receptors and is the principal chemomediator of pain impulses from the periphery to the central nervous system. The C-fibers or primary afferents of the peripheral nervous system are thought to be the main nociceptive neurons that secrete SP. Capsaicin is an agent that induces the release of SP from the C-fibers to depletion: with repeated administration, it may reduce neurogenic inflammation. However, capsaicin also activates C-fibers, causing acute pain, erythema and irritation and the use of such should be in conjunction with an agent to relieve these symptoms. Laughlin et al (U.S. Pat. No. 5,854,291) proposed the use of capsaicin in a formulation together with ingredients to neutralize the discomfort of capsaicin application.

The Croton species is a traditional medicine of the indigenous peoples of the Amazon River basin. The Jivaro Indians of central Perú use the latex (sap) in the treatment of various external dermal traumas (i.e. cuts, wounds, abrasions, bites, stings) as well as internally for the treatment of ulcers. A number of different Croton tree species (C. draconoides, C. erythrochilus, C. gossypifolius, C. lechleri, C. palanostigma, C. salutaris) found in South America produce this red latex commonly known as Sangre de grado or Sangre de drago (“Dragon's blood”).

Sangre de grado has been found to contain the alkaloid, taspine, which is believed to have anti-inflammatory activity (U.S. Pat. No. 3,694,557). Vaisberg et al reported that the hydrochloride salt of taspine is responsible for the cicatrizant activity of Croton lechleri. However, taspines wound healing capabilities are less significant effect than the parent material Sangre de grado.

Sangre de grado is an effective inhibitor of sensory afferent nerves. Inflammatory medications including prostaglandin E2, proteases and capsaicin are known activators of sensory afferent nerves, and augments many local tissue responses including vasodilation, pain perception, mast cell activation (i.e. itch, swelling, redness) and edema formation. Sensory afferent nerves innervate local blood vessels and mast cells (to release histamine and tryptase) and transmit the sensation of pain, itch, and/or discomfort to the central nervous system. Sangre de grado blocks the ability of these nerves to be activated by numerous agonists and as such, it is an inhibitor of neurogenic inflammation. As a result, the amplification of local tissue responses is blocked by Sangre de grado, as well as the brain's perception of troubling and discomforting symptoms. Further, these benefits occur immediately and do not require the weeks of sustained treatment that comonly leads to neurotransmitter depletion with capsaicin. Thus, by its inhibitory effect, Sangre de grado is broadly effective and a unique analgesic agent and antipruretic that provides fast relief of symptoms.

Sangre de grado its natural state however, is a viscous dark-red latex, high in aqueous components as well as discoloring agents, such as proanthrocyanidins. This shortcomming reduces its use directly or in combination with other compositions in commercial applications.

### SUMMARY OF THE INVENTION

Aspects of the invention are summarized below to aid in the understanding of embodiment(s) of the invention and the application. Yet, the invention is fully defined by the claims of the application.

Hemorrhoids are ubiquitous in humans. Current therapies for severe manifestations (i.e. surgery, ligation, cauterization) have changed little since recorded history. Topical medicaments are only marginally effective as they address limited aspects of the disease.

Ethnomedical treatments for human disease are primarily derived from native botanicals. In the case of the indigenous Amazonians, Sangre de grado or Sangre de drago latex (sap) from the bark of Croton species has been used in the treatment of dermal injury, which while effective, has undesirable properties, which limits its use in topical applications.

However, as disclosed herein, the Croton species latex can be concentrated by removing its aqueous components and extracted of discolorants resulting in an extract (Zangrado®) that is more biologically effective than the parent botanical, and also amenable to topical preparations while possessing an improved safety profile. Thus, the invention herein teaches that extracts and preparations of Croton species can be used as medicaments in the treatment of anorectal disease and offer significant benefit over the parent botanical or currently available medications. The present invention generally comprises methods and compositions for the treatment of anorectal disease in humans that reduces pain, swelling and itching of the affected tissues while simultaneously promoting resolution and healing. These methods and compositions contain botanical derivatives of the Croton species that retain the ability to inhibit the activation of sensory afferent nerves and neurogenic inflammation, reducing pain, edema, itching and associated symptoms, yet is amenable to topical formulation. The disclosure therefore results in effective methods and compositions that promote an analgesic effect and reduce the symptoms of discomfort while promoting the healing of hemorrhoids, anorectal fissures, and similar conditions. The further discovery of the biomechanism by which the Croton species latex acts leads to new applications for its use, which can be developed according to the disclosure herein. The compositions disclosed herein are further incorporated into a biologically active dosage unit for anorectal disease treatment compositions.

### BRIEF DESCRIPTION OF THE FIGURES

FIG. 1. The prototypical activator of sensory afferent nerves, capsaicin, was topically applied to the mucosal surface of the stomach in anesthetized rats and mucosal blood flow measured by a Laser Doppler Flow meter. The marked increase in mucosal blood flow induced by 300 μM capsaicin was prevented by either the parent material, Sangre de grado or its extract, Zangrado (CGO 110) at doses of 2 and 0.2 mg/ml, respectively, indicating that the organic extract retains the ability to effectively prevent the activation of sensory afferent nerves.

FIG. 2. Using a well-established ferret model of post-operative complications of itch induced by morphine, the pretreatment with Zangrado virtually abolished episodes of itch (* P<0.05). Thus, the extract offers significant improvement over the parent material or other preparations in the treatment of itch.

FIG. 3: In a well-defined model of edema, rats were pretreated with Zangrado prior to the intradermal injection of protease activated receptor-2 activating peptide (PAR₋₂AP) (SLIGRL-NH₂, 500 μg) into the footpad. As clearly indicated the decline in paw volume was more dramatic and sustained in the Zangrado treated group as compared to the placebo with only a single application, resulting in approximately 50% less edema.

FIG. 4: Using a standard model of pain response, rats were pretreated with Zangrado prior to footpad inoculation with PAR₂₋AP and subsequent subjection to a thermal stimulus. As determined in a Hargreave's apparatus, pretreatment with Zangrado prevented the induction of hyperalgesia, with the latency withdrawal time remaining at its baseline level despite PAR₂₋AP administration.

FIG. 5: A clinical trial of pest control workers in New Orleans revealed that those receiving a Zangrado containing balm received significant relief from insect bites, stings and other skin conditions as compared to placebo. Zangrado 1% balm relieved symptoms in all individuals for all applications. The Zangrado composition was preferred over placebo by all participants and relief was reported to occur (on average) in less than 2 minutes.

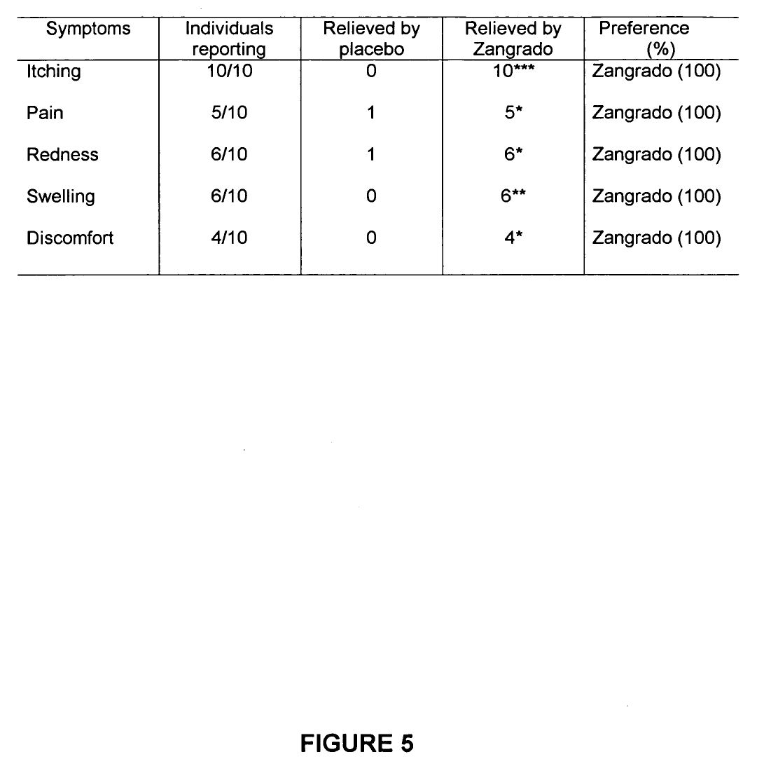

The invention described herein proposes that Sangre de grado or extracts thereof alone as the active constituent in an appropriate base vehicle or in combination with other known pharmaceutical compounds be utilized as a topical treatment in anorectal disease. The invention teaches that such an embodiment will improve current therapies by the combination of analgesic, antibacterial, anti-inflammatory, anti-secretory, anti-itch and cicatrizant properties. The use of said is appropriate in particular but not limited to those conditions associated with anorectal disease.

### DESCRIPTION OF AN EMBODIMENT

Extraction Procedures

According to one aspect of this invention, an organic extraction of Croton species latex concentrates its lipophilic components and reduces the hydrophilic proanthocyanidin content of the plant material. The invention demonstrates that Zangrado inhibits sensory afferent nerve activation and neurogenic inflammation, reducing pain, itch and inflammation more effectively than the parent material, representing an improvement in therapeutic activity.

According to one aspect of this invention, the extraction and removal of water through physical manipulation concentrates the biologically active components in Croton species latex. The invention teaches that these extracts are more therapeutically effective at lower concentrations than the parent material, representing a significant improvement in therapeutic potential and use.

Preferred methods to accomplish the aforementioned species Croton extractions have been previously described but it is contemplated that a skilled practitioner could device obvious variations of the procedures given the disclosure herein and the desired results.

Extraction Process 1.

Latex, or sap from Croton species is mixed with an organic solvent. The preferred organic solvent is ethyl acetate although other organic solvents can be used as would be obvious to the ordinarily skilled practitioner in light of the disclosure herein. In other embodiments, the preferred organic solvent is isopropanol, a chloroform/Methanol mixture, or an equivalent thereof. The organic solvent is added to the latex in a 1:1 proportion. In the preferred extraction process the solvent latex combination is agitated.

The preferred agitation method is stirring although other agitation methods are also contemplated to be effective. Following agitation, the mixture is settled, or allowed to settle into distinct phases including at least an organic layer and an aqueous layer. The organic phase or layer is comprised largely of solute lipophilic materials, representing the active constituent, and a significantly reduced quantity of proanthocyanidin components relative to the pre-agitation step. The organic layer is separated from the aqueous layer for further processing pursuant to the preferred extraction process.

Moreover, it is common to find a gel-like substance in the organic layer at the interface of the aqueous and organic layers. This gel substance is characterized as having a dark brown and purple color and comprises hydrophilic constituents trapped with water. In the preferred process the gel substance is processed further to separate any active lipophilic constituents from the hydrophilic constituents. The preferred manner of processing the gel substance is the addition of a drying agent to the organic layer or the gel substance. The preferred drying agent is magnesium sulfate in a concentration of 0.5-5 g/L of contaminant gel. It is contemplated that other equivalent drying agents at relative effective concentrations would also be effective and would be obvious to the ordinarily skilled practitioner in light of the disclosure herein and with undue experimentation.

The addition of the drying agent results in a precipitant, which traps water and hydrophilic constituents or water-based colored chemical contaminants. The precipitant can be readily separated from the hydrophilic constituents by filtration or other techniques known to separate precipitants. Actual laboratory procedures achieved acceptable results using a Whatman #4 filter paper or an equivalent.

The steps of organic extraction, mixing with a drying agent and filtration may be repeated up to three times to accomplish a thorough extraction of the active lipophilic constituents. At this point in the process, the lipophilic materials are solutes contained within the organic solvent, which are concentrated by evaporation of the solvent by one of several procedures, such as vacuum drying, freeze drying or heating. Actual heating up to 60 degrees Celsius produced acceptable drying results.

The organic layer composition thus processed is rich in lipophilic materials but largely clear of hydrophilic contaminants. Following the extraction process, the color of the organic layer can be characterized as a rose. Moreover, the reduced proanthocyanidin content is quantifiable spectrophotometrically. Relative absorbance of the extraction in the visible spectrum was compared to the absorbency peak of the parent latex (414 nm) in the visible range.

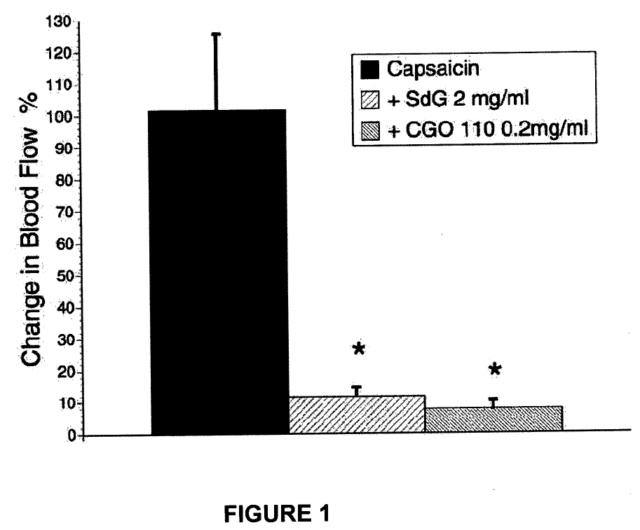

At a concentration of 1 mg of extracted latex to 1 mL of water the disclosed process yielding the extraction (CGO 110) results in a 4.3 fold reduction in absorbance at 414 nm, as indicated in FIG. 1. This assessment was repeated 9 times with similar results achieved (significance difference P<0.0001, as denoted by the “*”). Similarly when sangre de grado or the extraction (CGO 110) at a concentrations of 200 μg per mL of aloe vera gel were applied to aloe vera gel to mimic their administration as topical products, there was also a significantly lower color response with the extracted sangre de grado, CGO 110 vs. the parent botanical (* P<0.0001). See FIG. 1. Estimates from the absorbency measurements indicate that the proanthocyanidin content was reduced by at least 90% relative to the nonextracted parent latex.

Extraction Process 2.

The latex from the Croton species is dried to its residual solid matter by methods such as heating, air-drying, vacuum or freeze-drying. The dried latex is rich in proanthocyanidin compounds and therefore characterized by a dark burgundy color. To the dried latex matter the organic solvent, ethyl acetate or an equivalent, is added. The dried latex and organic solvent mixture is agitated and the organic solvent is removed for further processing according to the procedure described in Process 1. This process may be repeated up to three times to accomplish a thorough extraction all lipophilic materials in the organic layer and solvent. If any water bearing contaminants are present, the addition of drying agent followed by filtration as noted above, will remove these contaminants. Removing the ethyl acetate through various methods including heating, air-drying, vacuum or freeze-drying then isolates the solutes contained within this organic extract.

The extraction thus processed according to the disclosed processes is characterized by a significant reduction of proanthocyanidin compounds. The reduction of the proanthocyanidin compounds leaves the extraction significantly diminished in color producing compounds and yet amenable to health care applications.

Effects of Zangrado on Sensory Afferents

Sensory afferent nerves mediate the sensations of pain, itch, cough and nausea and when activated by capsaicin, lead to a multitude of responses including vasodilation, inflammation, edema, and pain and itching. Using a well-established rat model, the mucosal surface of the stomach in anesthetized animals was treated with 300 μM capsaicin and mucosal blood flow measured by a laser Doppler flow meter.

As indicated in FIG. 1, the marked increase in capsaicin-induced mucosal blood flow was prevented by either the parent material or its organic extract Zangrado at doses of 2 and 0.2 mg/ml, respectively. Thus, the organic extract described in this application retains the ability to effectively prevent the activation of sensory afferent nerves and offers significant benefit over the parent material.

Effects of Zangrado on Morphine-Induced Itch and Emesis

Using a well-established ferret model of post-operative complications of nausea, emesis and itch induced by morphine, the extraction Zangrado was administered intraperitoneally (3 mg/kg) to ferrets 15 minutes prior to the administration of morphine-6-glucuronide (M6G), known to promote itching, retching and vomiting. The animals were monitored for sixty minutes.

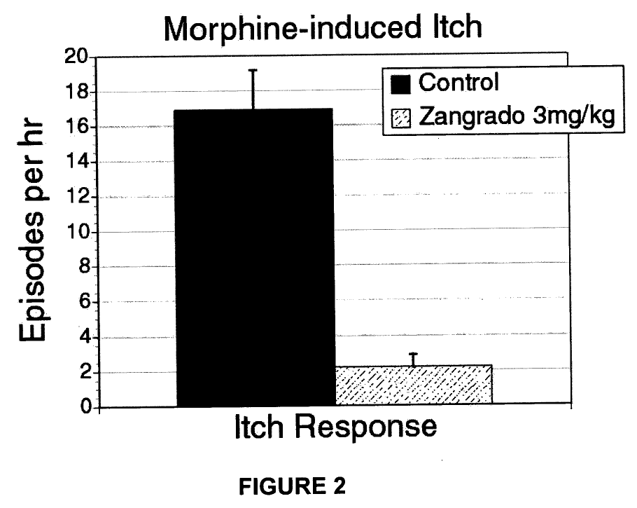

As shown in FIG. 2, the M6G caused a significant number of itching episodes as indicated by licking responses which was greatly attenuated in Zangrado treated animals (* P<0.05). Thus, the extract offers significant improvement over the parent material or other preparations in the treatment of itch.

Reduction of Edema

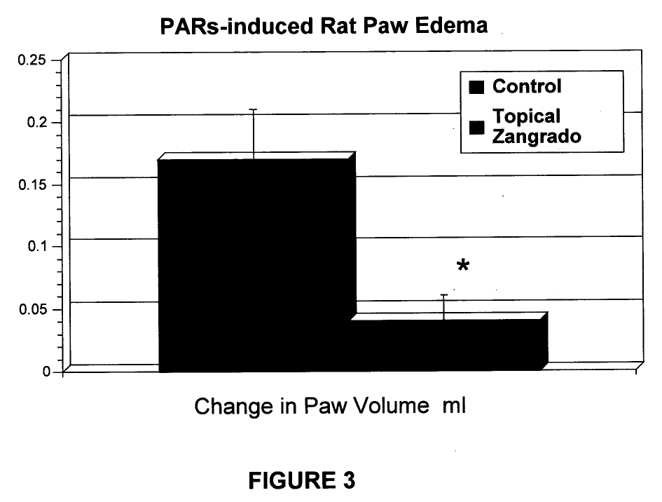

Spraque-Dawley rats (250 g) were anesthetized with sodium pentobarbital. Twenty-two minutes prior to the intradermal injection of protease activated receptor-2 activating peptide (PAR₋₂AP) (SLIGRL-NH₂, 500 μg) into the rat footpad, rats received either 40 mg of placebo balm or a balm containing Zangrado™ (1% Sangre de grado extract) topically to the footpad. Basal paw volume was measured before PAR₂₋AP administration, and every subsequent hour for a total of six hours. An individual unaware of the treatment groups measured paw volume using a hydroplethysmometer bath. As indicated in FIG. 3, the decline in paw volume was more dramatic and sustained in the SdG (Zangrado) treated group as compared to the placebo with only a single application, resulting in approximately 50% less edema.

Reduction of Hyperalgesia

Rats were treated in a manner similar to previously descriptions except that the PAR₂₋AP dosage was reduced (50 μg) to avoid complications from edema. Fifteen minutes prior to the injection, the rats were divided into two groups and either treated topically with 40 mg of a placebo balm or a balm containing 1% Zangrado. Paw withdrawal latency time to a thermal stimulus, as determined in a Hargreave's apparatus, was used as the index of pain sensitivity. Withdrawal times were determined in each group prior to PAR₂₋AP administration (basal), and then 30 and 60 minutes after administration. A reduction in the latency withdrawal time is used as an index of hyperalgesia.

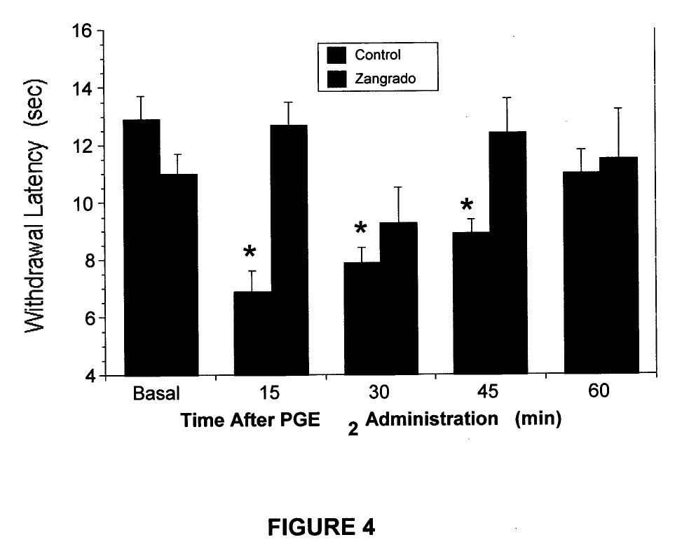

As show in FIG. 4, the intradermal injection of PAR₂₋AP resulted in a decrease in the latency withdrawal period to a heat source indicative of a state of hyperalgesia. Pretreatment with Zangrado balm 1% prevented the induction of hyperalgesia, with the latency withdrawal time remaining at its baseline level despite PAR₂₋AP administration. The Zangrado 1% balm did not affect the withdrawal latency in rats that did not receive the PAR₂₋AP injection (data not shown) indicating that it was not acting as an anesthetic.

Hyperalgesia was also induced by intradermal prostaglandin E₂ (PGE₂), which is thought to induce an increased sensitivity to pain perception by raising the resting potential of sensory afferent nerve fibers. In these experiments, intradermal PGE₂ resulted in a significant reduction in paw withdrawal time and this effect was blocked by a single topical administration of Zangrado 1% balm.

Clinical Evaluation of SDG-Based Balm

An evaluation of Zangrado balm for relief of the symptoms associated with insect bites, stings and other skin conditions was conducted in pest control workers in New Orleans, La., USA. Participants compared the effects of a placebo balm with a balm containing 1% Zangrado over a 3 month period.

As noted in Table 1, itching was the predominant symptom, reflecting the preponderance of fire ant bites among the workers. The placebo balm offered relief in only two cases whereas the Zangrado 1% balm relieved symptoms in all individuals for all applications. The Zangrado 1% balm was preferred over placebo balm by all participants and relief was reported to occur in less than 2 minutes, on average.

The invention described herein proposes that Sangre de grado or extracts thereof alone as the active constituent in an appropriate base vehicle or in combination with other known pharmaceutical compounds be utilized as a topical treatment in anorectal disease. The invention teaches that such an embodiment will improve current therapies by the combination of analgesic, antibacterial, anti-inflammatory, anti-secretory, anti-itch and cicatrizant properties. The use of said is appropriate in particular but not limited to those conditions associated with anorectal disease.

Although the invention has been described in detail with reference to one or more particular preferred embodiments, persons possessing ordinary skill in the art to which this invention pertains will appreciate that various modifications and enhancements may be made without departing from the spirit and scope of the claims that follow.

1. A method for treating anorectal disease comprising the application of an extract of the Croton species having substantially reduced proanthocyanidin content and concentrated lipophilic constituents.

2. The method in claim 1 wherein the application further comprises the application of an anti-inflammatory.

3. (Cancelled)

4. The method of claim 1 wherein the lipophilic constituents are extracted from plant material that is from the family Euphorbaciae.

5. The method in claim 1 wherein the extract is of the latex of the Croton species containing less than 10% water.

6. The method in claim 1 wherein the extract is of the Croton species in a final concentration of 20 to 300 micrograms per milliliter.

7. The method in claim 1 where the extract is in an amount by weight between 0.05% and 40.0%.

8. (Cancelled)

9. (Cancelled)

10. The method in claim 2 wherein the anti-inflammatory agent is at least one selected from the group consisting of; betamethasone, clobetasol, desoximetasone, dexamethasone, fluocinolone, flucinomide, cortisone, hydrocortisone, and triamcinolone.

11. (Cancelled)

12. (Cancelled)

13. (Cancelled)

14. (Cancelled)

15. (Cancelled)

16. (Cancelled)

17. (Cancelled)

18. A pharmaceutical dosage unit comprising an extract of family Euphorbaciae for the treatment of symptoms associated with anorectal disease and reduced proanthocyanidin content and concentrated lipophilic consituants.

19. The pharmaceutical dosage unit in claim 18 wherein the symptoms are selected from the group consisting of; pruritis, edema, and hyperalgeisa.

20. The pharmaceutical dosage unit in claim 18 that inhibits the activation of sensory afferent nerves.

21. The pharmaceutical dosage unit in claim 18 wherein the extract is of the Croton species in a final concentration of at least 100 micrograms per milliliter of application area of skin.

22. The pharmaceutical dosage unit in claim 18 wherein the extract is included as an ingredient for a topically applied hemorrhoid treatment.

23. The pharmaceutical dosage unit in claim 18 wherein the extract is applied with an anti-inflammatory.

24. The pharmaceutical dosage unit in claim 23 wherein the anti-inflammatory is at least one selected from the group consisting of; betamethasone, clobetasol, desoximetasone, dexamethasone, fluocinolone, flucinomide, cortisone, hydrocortisone, and triamcinolone.

25. The method in claim 1 wherein the extract deters the activation of sensory afferent nerves.

26. The method in claim 1 wherein the extract is of the Croton species in a final concentration of at least 100 micrograms per milliliter of application area of skin.

27. The method in claim 1 wherein the extract is included as an ingredient for a topically applied hemorrhoid treatment.
